(19) United States
(12) Patent Application Publication
(10) Pub. No.: US 2019/0224041 A1
(43) Pub. Date: Jul. 25, 2019

(54) FEEDBACK MECHANISM IN A PESSARY

(71) Applicant: GYNOTECH LTD., Holon (IL)

(72) Inventors: Abraham NISSENKORN, Holon (IL); Meir TEICHNER, Yoqneam-Moshava (IL); Arieh BAR ZEEV, Raanana (IL)

(21) Appl. No.: 16/327,967

(22) PCT Filed: Aug. 23, 2017
(86) PCT No.: PCT/IL2017/050941

§ 371 Date: Feb. 25, 2019

Related U.S. Application Data
(60) Provisional application No. 62/379,273, filed on Aug. 25, 2016.
(60) Provisional application No. 62/461,230, filed on Feb. 21, 2017.

(51) Int. Cl. A61F 6/08; A61F 2/00; A61B 5/03

(52) U.S. Cl. CPC A61F 6/08; A61F 2/005; A61F 2250/0004; A61F 2250/0002; A61F 2230/0065; A61B 5/036

(57) ABSTRACT

A feedback mechanism for an adjustable vaginal pessary adapted to provide real-time adjustments in order to provide support to vaginal walls of a patient suffering of pelvic genital organ prolapse and female urinary distress, especially stress incontinence; comprising: at least one pressure sensor; at least one computer comprising at least one microprocessor, at least one memory, at least one input/output (I/O), at least one wireless adaptor, at least one transmitter; at least one motor; at least one battery; at least one pessary adjusting mechanism; wherein a change in pressure recorded by said at least one pressure sensor activates said feedback mechanism changing configuration of said adjustable pessary. Furthermore, the present invention utilizes the pessary mechanism as a rape salvation pessary.

This application is a continuation of U.S. provisional application 62/318,241 and U.S. provisional application 62/461,230 WHICH ARE INCORPORATED HEREIN ENTIRELY.

### FIELD of the invention

This invention relates in general to vaginal pessaries used for treating pelvic genital organ prolapse and female urinary incontinence, including stress incontinence; and more specific to a feedback mechanism in the pessary which enables said pessary to change its configuration in the presence of a change in the intravaginal pressure. Furthermore, the present invention utilizes the pessary mechanism as a rape salvation pessary.

### BACKGROUND OF THE INVENTION

Female genital prolapse (or vaginal prolapse or pelvic organ prolapse) is characterized by a portion of the vaginal canal protruding (prolapsing) from their normal position. The condition usually occurs when the pelvic floor is weakened and, with further gravity, when it collapses as a result of childbirth, heavy lifting, chronic cough and other, which results in increasing intra-abdominal pressure, which can tear soft tissues, i.e. herniating fascia membranes, muscles and others, so that the vaginal wall collapses, resulting in cystocele, rectocele and other components of the vaginal walls, uterine prolapse in different combinations (https://en.wikipedia.org/wiki/Female_genital_prolapse—incorporated herein as reference).

Results of a new survey commissioned by the American Foundation for Urologic Disease (AFUD) and conducted by Harris Interactive show that 57% of women surveyed have experienced symptoms of stress urinary incontinence (SUI), a medical condition that results in accidental uncontrolled/involuntary urine leakage when they sneeze, cough, laugh, lift, or exercise. Among those surveyed who described SUI symptoms or have been diagnosed with the condition, more than one in four say that urine leakage affects their lives every day. SUI is the most prevalent form of urinary incontinence, but women rarely discuss their symptoms with a healthcare professional. Some research showed that most women go to the doctor about 5 years after the appearance of the symptoms.

In general, urinary incontinence affects about 30% of the female population in the United States and worldwide. It can lead to a profound deterioration in the quality of life of affected women. In the short term, avoidance behaviors are common. In the long term, other medical issues may complicate the condition, including depression, skin disorders, hygienic problems, and fractures resulting from falls related to the absence of a commode in close proximity to the patient, especially at night.

As urine must exit the bladder through the urethra, techniques are available to mechanically obstruct this outflow during stress by lifting the area into the influence of the intraabdominal pressure zone. In the woman who only loses urine while exercising, a tampon of the largest size that is comfortable may provide this obstruction. Continence tampons are available. Any stress-incontinent patient may be fitted with a continence pessary designed for placement under the urethra. Both devices provide temporary urethral occlusion by pushing against the urethra during stress episodes or by lifting the bladder sphincter into the intraabdominal pressure zone (somewhat like stepping on an active garden hose). Other mechanical devices are designed to be placed into the urethra as a plug or to be glued to the urethral meatus to block urine exit from the urethra. The former device, however, causes different side effects and frequent urinary tract infections.

A pessary is a device placed into the vagina to support the prolapsing vaginal walls or to prevent urinary incontinence (Technical Update on Pessary Use, J Obstet Gynaecol Can 2013; 35(7 eSuppl):S1-S11—incorporated herein as reference). Pessaries have the distinct advantage of being minimally invasive, minimally irritating, minimally disturbing, etc., and they provide immediate relief of symptoms. Pessaries are made primarily of medical grade silicone; only the largest sizes are made of silicone or rubber, and some of them comprise surgical steel within. This has the advantage of making them inert and less likely to have an odor or cause an allergic reaction. Pessaries at present are used for the treatment of prolapse can be classified as support pessaries or space-occupying pessaries. The support pessaries sit in the posterior fornix and generally rest under the pubic bone and/or pelvic floor (inside the vagina). The commonly used types include ring pessaries (with or without diaphragms, Figure la) and the Shaatz pessaries (FIG. 1b). The space-occupying pessaries include the cube (FIG. 1c), Inflatoball (FIG. 1d), and donut (FIG. 1e) pessaries. The cube works by bringing the vaginal walls towards the midline, and the others occupy a larger vaginal space than the introitus. They are most often used for more severe prolapse. The commonly used Gellhorn pessary (FIG. 1f) works as a combination of these methods. Incontinence pessaries are often designed as support pessaries with extra support anteriorly (FIG. 1g) to elevate and slightly constrict the urethra. The ring incontinence pessary (FIG. 1h) and the incontinence dish (FIG. 1i) are specifically designed to treat stress urinary incontinence. If a woman develops stress incontinence after being fitted with a prolapse pessary, switching to an incontinence pessary may be beneficial (FIGS. 1a-i from CooperSurgical Inc., incorporated herein as reference). Some pessaries have been specifically designed to treat urinary stress incontinence. These include the ring pessary with support and knob (FIG. 1g), the incontinence ring (FIG. 1h), the incontinence dish (FIG. 1i). They theoretically appear to stabilize the urethra and increase urethral resistance. Initial successful fitting varies between 60% and 92%, with an incontinence ring. Continued use drops to 55% by 6 months. By 1 year, the overall continuation may be as low as 16%, and most women discontinued use because of lack of efficacy. Reasons for discontinuation included persistent incontinence, pessary falling out, or pain and bleeding. The main reason for that discontinuation is the fact that at present, the pessaries are of unchangeable non-adjustable sizes and diameter, and therefore if a patient changes its physical activity, therefore changing the intra-abdominal pressure, the pessary may not fit anymore to the new conditions, and the patient may need to replace the pessary by a different size pessary.

In US patent application 62/318,241, we described a novel pessary which provides an easy-to-use adjustable pessary that can preserve a circular geometry during its adjustment in order to provide a better support during any type of situation, performance or intra-abdominal pressure and preventing further migration or prolapse. Another disclosure of the aforementioned invention provides the pessary with a folding mechanism that provides even further support and preventing further migration or prolapse.

Another problem, which was not disclosed before is that, in some cases, the patient is not in close proximity to a place where she can adjust the pessary or she is involved in some activity that does not allow stopping and adjusting the pessary, like daily basis activities like going to the gym, lifting, cleaning, and more; and obviously during more intensive activities like marathons, rock climbing, swimming, etc. In those cases, there is a need for an automated mechanism that allows the pessary to adapt according to the necessities.

U.S. Pat. No. 6,039,701 discloses an apparatus for measuring cervical diameter comprises a support structure and measurement devices for detecting changes in cervical diameter, either directly or indirectly through changes in the size of the support structure. The support structure may conform to a cervical surface, typically being a peripherally expansible lumen or expansible structure. Alternatively, the support structure may engage the vaginal wall or fornices. Measurement devices may include gages which determine change in sizes of an expansible loop, electronic devices for measuring changes in transmitted or reflected energy, or combinations thereof. The devices are suitable for use on ambulatory patients and in out-patient situations. The apparatus comprises several sensors, like a magnetic sensor, tracking sensors, strain sensors and others.

U.S. Pat. No. 9,314,227 discloses an apparatus for determining at least one parameter in the vaginal channel of a user. The apparatus comprises a flexible pessary and a measurement unit comprising at least one sensor for measuring the at least one vaginal parameter. The measurement unit is reversibly mountable to the flexible pessary. The apparatus comprises several sensors, like a temperature sensor.

Patent application US20140073879 discloses a system is disclosed for long term, continuous, monitoring of pregnant mammals particularly to detect the onset of preterm labor. For example, the system enables communication between sensors and a collection unit for transmittal to a remote unit for display and monitoring of the collected data by a clinician while the individual is ambulatory and not in the presence of the clinician. The system comprises sensors that measures at least one of electrical impedance, pH and temperature.

Furthermore, the present application provides a rape salvation pessary by using the same anti-prolapse urinary incontinence mechanism.

Regarding this matter, U.S. Pat. No. 4,508,114 discloses an anti-rape device that is adapted to be worn in the vaginal cavity of a female. The device comprises a hollow housing with adhesive means on the interior of the housing to adhere to any rapist and an irritant-containing pouch positioned within the housing which ruptures upon forceful contact with the rapist. The adhesive means ensures the device will adhere to the rapist thereby causing continued discomfort.

U.S. Pat. No. 4,192,066 discloses an anti-rape defensive weapon comprising a barbed shank similar to a fish-hook barb carried in a barrel like hand grip arranged to expose the barbed shank when the device is pressed against the flesh of an assailant. A particular feature provides for the separation of the shank lodged in the assailant.

The aforementioned pessaries do not disclose or suggest the disclosure of the present invention, which is to provide a pessary comprising pressure sensors connected to an actuator that change the configuration of the pessary automatically. And further, they do not disclose the use of the same mechanism as a rape salvation device.

### SUMMARY OF THE INVENTION

It is hence a scope of the present invention to disclose an adjustable vaginal pessary adapted to provide support to vaginal walls of a patient comprising within: a pessary expanding and contracting mechanism; at least one intravaginal pressure sensor interconnected to said pessary; at least one computer for processing data from said pressure sensor interconnected to said sensor; wherein said pessary expanding and contracting mechanism is provided with at least one motor for expanding and contracting said reversibly expandable pessary; further wherein said computer is configured to translate said pressure data into programmed instructions for expanding or contracting the configuration of said adjustable pessary in predetermined feedbacked manner.

It is hence another scope of the present invention to disclose the adjustable vaginal pessary, wherein said computer comprises at least one microprocessor, at least one memory, at least one input/output (I/O), at least one wireless adaptor and at least one transmitter.

It is hence another scope of the present invention to disclose the adjustable vaginal pessary, wherein said at least one pressure sensor is selected from the group consisting of: absolute pressure sensor, gauge pressure sensor, vacuum pressure sensor, differential pressure sensor, sealed pressure sensor, and any combination thereof.

It is hence another scope of the present invention to disclose the adjustable vaginal pessary, wherein said at least one pressure sensor is selected from the group of pressure-sensing technology consisting of: piezoresistive strain gauge, capacitive, electromagnetic, piezoelectric, optical, potentiometric, resonant, thermal, ionization, and any combination thereof.

It is hence another scope of the present invention to disclose the adjustable vaginal pessary, wherein said at least one memory further comprises a program recorded therein to be executed by said at least one microprocessor.

It is hence another scope of the present invention to disclose the adjustable vaginal pessary, wherein said at least one pessary adjusting mechanism is selected from the group consisting of pneumatic, hydraulic, mechanical, ratchet, telescoping.

It is hence another scope of the present invention to disclose the adjustable vaginal pessary, wherein said adjustable vaginal pessary further comprises a battery.

It is hence another scope of the present invention to disclose the adjustable vaginal pessary, wherein said battery is a rechargeable battery.

It is hence another scope of the present invention to disclose the adjustable vaginal pessary, wherein said rechargeable battery is a kinetic rechargeable battery.

It is hence another scope of the present invention to disclose the adjustable vaginal pessary, wherein the actions made by said feedback mechanism are programmable.

It is hence another scope of the present invention to disclose the adjustable vaginal pessary, wherein the programming is done physically via a digital handle or wirelessly via a smartphone, tablet or a dedicated remote control.

It is hence another scope of the present invention to disclose the adjustable vaginal pessary, wherein said changing configuration of said adjustable pessary occurs in about one second.

It is hence another scope of the present invention to disclose the adjustable vaginal pessary, wherein said feedback mechanism increases/decreases the ring's diameter in jumps of at least 1 millimeter (mm).

It is hence another scope of the present invention to disclose the adjustable vaginal pessary, wherein the increase/decrease of the ring's diameter can be set by means of said actuator.

It is hence another scope of the present invention to disclose the adjustable vaginal pessary, wherein said feedback mechanism is encapsulated in a protective insulating biocompatible material.

It. is hence another scope of the present invention to disclose the adjustable vaginal pessary, wherein said pessary is used as a rape salvation device.

It is hence a scope of the present invention to disclose a feedback mechanism for an adjustable vaginal pessary adapted to provide real-time adjustments in order to provide support to vaginal walls of a patient comprising: at least one pressure sensor interconnected to said vaginal pessary for detecting intravaginal pressure; at least one computer comprising at least one microprocessor, at least one memory, at least one input/output (I/O), at least one wireless adaptor, at least one transmitter; said at least one computer interconnected to said at least one pressure sensor and configured for processing data from said pressure sensor; at least one motor interconnected to said at least one computer; at least one battery interconnected to said at least one pressure sensor, said at least one computer and said at least one motor; at least one pessary adjusting mechanism interconnected to said at least one motor; wherein said computer is configured to translate said pressure data into programmed instructions for expanding or contracting the configuration of said adjustable pessary in predetermined manner.

It is hence another scope of the present invention to disclose the feedback mechanism, wherein said at least one pressure sensor is selected from the group consisting of: absolute pressure sensor, gauge pressure sensor, vacuum pressure sensor, differential pressure sensor, sealed pressure sensor, and any combination thereof.

It is hence another scope of the present invention to disclose the feedback mechanism, wherein said at least one pressure sensor is selected from the group of pressure-sensing technology consisting of: piezoresistive strain gauge, capacitive, electromagnetic, piezoelectric, optical, potentiometric, resonant, thermal, ionization, and any combination thereof.

It is hence another scope of the present invention to disclose the feedback mechanism, wherein said at least one memory further comprises a program recorded therein to be executed by said at least one microprocessor.

It is hence another scope of the present invention to disclose the feedback mechanism, wherein said at least one pessary adjusting mechanism is selected from the group consisting of pneumatic, hydraulic, mechanical, ratchet, telescoping.

It is hence another scope of the present invention to disclose the feedback mechanism, wherein said battery is a rechargeable battery.

It is hence another scope of the present invention to disclose the feedback mechanism, wherein said rechargeable battery is a kinetic rechargeable battery.

It is hence another scope of the present invention to disclose the feedback mechanism, wherein the actions made by said feedback mechanism are programmable.

It is hence another scope of the present invention to disclose the feedback mechanism, wherein the programming is done physically via a digital handle or wirelessly via a smartphone, tablet or a dedicated remote control.

It is hence another scope of the present invention to disclose the feedback mechanism, wherein said changing configuration of said adjustable pessary occurs in about one second.

It is hence another scope of the present invention to disclose the feedback mechanism, wherein said feedback mechanism increases/decreases the ring's diameter in jumps of at least 1 millimeter (mm).

It is hence another scope of the present invention to disclose the feedback mechanism, wherein the increase/decrease of the ring's diameter can be set by means of said actuator.

It is hence another scope of the present invention to disclose the feedback mechanism, wherein said feedback mechanism is encapsulated in a protective insulating biocompatible material.

It is hence another scope of the present invention to disclose the feedback mechanism, wherein said feedback mechanism is used as a rape salvation device.

It is hence a scope of the present invention to disclose a method for providing real-time adjustments to an adjustable pessary, said method comprising the steps of: acquiring an adjustable vaginal pessary comprising an inner mechanism, an outer envelope, an actuator and a feedback mechanism: said inner mechanism comprises: a split ring main body having a diameter D; an opening/closing mechanism interconnected to said split ring; said outer envelope comprises: a biocompatible material envelope; a sleeve made of biocompatible material; said feedback mechanism comprises: at least one pressure sensor; at least one computer comprising at least one microprocessor, at least one memory, at least one input/output (I/O), at least one wireless adaptor, at least one transmitter; said at least one computer interconnected to said at least one pressure sensor; at least one motor interconnected to said at least one computer; at least one battery interconnected to said at least one pressure sensor, said at least one computer and said at least one motor; at least one pessary adjusting mechanism interconnected to said at least one motor; inserting said adjustable vaginal pessary into the vaginal tract; adjusting said adjustable vaginal pessary by actuating said opening/closing mechanism by means of said actuator therefore increasing/decreasing said diameter D of said split ring until it meets the necessities of said patient; wherein during said step of adjusting said adjustable vaginal pessary by actuating said opening/closing mechanism said split ring preserves a circular geometry; setting said feedback mechanism by means of said actuator.

It is hence another scope of the present invention to disclose the method, wherein between said step (b) and said step (c) further comprises a step of inserting said actuator in said sleeve in order to reach said opening/closing mechanism and said feedback mechanism.

It is hence another scope of the present invention to disclose the method, wherein said method is adapted for use in treating pelvic organ prolapse.

It is hence another scope of the present invention to disclose the method, wherein said method is adapted for use in treating stress incontinence.

It is hence another scope of the present invention to disclose the method, wherein said step of adjusting said adjustable vaginal pessary by actuating said opening/closing mechanism does not increase or decrease the overall volume of said pessary.

It is hence another scope of the present invention to disclose the method, wherein said inner mechanism comprises pneumatic means for adjusting said ring.

It is hence another scope of the present invention to disclose the method, wherein said inner mechanism comprises hydraulic means for adjusting said ring.

It is hence another scope of the present invention to disclose the method, wherein said inner mechanism comprises mechanical means for adjusting said ring.

It is hence another scope of the present invention to disclose the method, wherein said mechanical means is a ratchet mechanism.

It is hence another scope of the present invention to disclose the method, wherein said mechanical means is a telescoping mechanism.

It is hence another scope of the present invention to disclose the method, wherein said at least one pressure sensor is selected from the group consisting of: absolute pressure sensor, gauge pressure sensor, vacuum pressure sensor, differential pressure sensor, sealed pressure sensor, and any combination thereof.

It is hence another scope of the present invention to disclose the method, wherein said at least one pressure sensor is selected from the group of pressure-sensing technology consisting of: piezoresistive strain gauge, capacitive, electromagnetic, piezoelectric, optical, potentiometric, resonant, thermal, ionization, and any combination thereof.

It is hence another scope of the present invention to disclose the method, wherein said at least one memory further comprises a program recorded therein to be executed by said at least one microprocessor.

It is hence another scope of the present invention to disclose the method, wherein said at least one pessary adjusting mechanism is selected from the group consisting of pneumatic, hydraulic, mechanical, ratchet, telescoping.

It is hence another scope of the present invention to disclose the method, wherein said battery is a rechargeable battery.

It is hence another scope of the present invention to disclose the method, wherein said rechargeable battery is a kinetic rechargeable battery.

It is hence another scope of the present invention to disclose the method, wherein the actions made by said feedback mechanism are programmable.

It is hence another scope of the present invention to disclose the method, wherein the programming is done physically via a digital handle or wirelessly via a smartphone, tablet or a dedicated remote control.

It is hence another scope of the present invention to disclose the method, wherein said actuator is a digital actuator.

It is hence another scope of the present invention to disclose the method, wherein said step of setting said feedback mechanism is done physically via a digital handle or wirelessly via a smartphone, tablet or a dedicated remote control.

It is hence another scope of the present invention to disclose the method, wherein said feedback mechanism changes the configuration of said adjustable pessary in about one second.

It is hence another scope of the present invention to disclose the method, wherein said feedback mechanism increases/decreases the ring's diameter in jumps of at least 1 millimeter (mm).

It is hence another scope of the present invention to disclose the method, wherein the increase/decrease of the ring's diameter can be set by means of said actuator.

It is hence another scope of the present invention to disclose the method, further comprising a step of providing said feedback mechanism and said inner mechanism encapsulated in a protective insulating biocompatible material.

It is hence another scope of the present invention to disclose the method wherein said computer further comprises instructions executable by said microprocessor to: actuating said opening/closing mechanism by means of said actuator therefore decreasing said diameter D of said split ring to a specified diameter, if said at least one pressure sensor measures a great increase in pressure; locking said opening/closing mechanism until further notice; thereby actuating as a rape salvation device.

It is hence a scope of the present invention to disclose a method for providing real-time adjustments to an adjustable pessary, said method comprising the steps of: acquiring an adjustable vaginal pessary comprising an inner mechanism, an outer envelope, an actuator and a feedback mechanism: said inner mechanism comprises: a split ring main body having a diameter D; an opening/closing mechanism interconnected to said split ring; a reversible pivot mechanism interconnected to said split ring; said outer envelope comprises: a biocompatible material envelope; a sleeve made of biocompatible material; said feedback mechanism comprises: at least one pressure sensor; at least one computer comprising at least one microprocessor, at least one memory, at least one input/output (I/O), at least one wireless adaptor, at least one transmitter; said at least one computer interconnected to said at least one pressure sensor; at least one motor interconnected to said at least one computer; at least one battery interconnected to said at least one pressure sensor, said at least one computer and said at least one motor; at least one pessary adjusting mechanism interconnected to said at least one motor; inserting said adjustable vaginal pessary into the vaginal tract; adjusting said adjustable vaginal pessary by actuating said opening/closing mechanism and said reversible pivot mechanism by means of said actuator therefore increasing/decreasing said diameter D of said split ring until it meets the necessities of said patient; wherein during said step of adjusting said adjustable vaginal pessary by actuating said opening/closing mechanism said split ring preserves a circular geometry; further wherein during said step of adjusting said adjustable vaginal pessary by actuating said reversible pivot mechanism, said reversible pivot mechanism confers and preserves an angle to said split ring; setting said feedback mechanism by means of said actuator.

It is hence another scope of the present invention to disclose the method, wherein said actuator actuates said opening/closing mechanism and said reversible pivot mechanism independently.

It is hence another scope of the present invention to disclose the method, wherein between said step (b) and said step (c) further comprises a step of inserting said actuator in said sleeve in order to reach said opening/closing mechanism and said reversible pivot mechanism.

It is hence another scope of the present invention to disclose the method, wherein said method is adapted for use in treating pelvic organ prolapse.

It is hence another scope of the present invention to disclose the method, wherein said method is adapted for use in treating stress incontinence.

It is hence another scope of the present invention to disclose the method, wherein said step of adjusting said adjustable vaginal pessary by actuating said opening/closing mechanism does not increase or decrease the overall volume of said pessary.

It is hence another scope of the present invention to disclose the method, wherein said inner mechanism comprises pneumatic means for adjusting said ring.

It is hence another scope of the present invention to disclose the method, wherein said inner mechanism comprises hydraulic means for adjusting said ring.

It is hence another scope of the present invention to disclose the method, wherein said inner mechanism comprises mechanical means for adjusting said ring.

It is hence another scope of the present invention to disclose the method, wherein said mechanical means is a ratchet mechanism.

It is hence another scope of the present invention to disclose the method, wherein said mechanical means is a telescoping mechanism.

It is hence another scope of the present invention to disclose the method, wherein said at least one pressure sensor is selected from the group consisting of: absolute pressure sensor, gauge pressure sensor, vacuum pressure sensor, differential pressure sensor, sealed pressure sensor, and any combination thereof.

It is hence another scope of the present invention to disclose the method, wherein at least one pressure sensor is selected from the group of pressure-sensing technology consisting of: piezoresistive strain gauge, capacitive, electromagnetic, piezoelectric, optical, potentiometric, resonant, thermal, ionization, and any combination thereof.

It is hence another scope of the present invention to disclose the method, wherein said at least one memory further comprises a program recorded therein to be executed by said at least one microprocessor.

It is hence another scope of the present invention to disclose the method, wherein said at least one pessary adjusting mechanism is selected from the group consisting of pneumatic, hydraulic, mechanical, ratchet, telescoping.

It is hence another scope of the present invention to disclose the method, wherein said battery is a rechargeable battery.

It is hence another scope of the present invention to disclose the method, wherein said rechargeable battery is a kinetic rechargeable battery.

It is hence another scope of the present invention to disclose the method, wherein the actions made by said feedback mechanism are programmable.

It is hence another scope of the present invention to disclose the method, wherein the programming is done physically via a digital handle or wirelessly via a smartphone, tablet or a dedicated remote control.

It is hence another scope of the present invention to disclose the method, wherein said actuator is a digital actuator.

It is hence another scope of the present invention to disclose the method, wherein said step of setting said feedback mechanism is done physically via a digital handle or wirelessly via a smartphone, tablet or a dedicated remote control.

It is hence another scope of the present invention to disclose the method, wherein said feedback mechanism changes the configuration of said adjustable pessary in about one second.

It is hence another scope of the present invention to disclose the method, wherein said feedback mechanism increases/decreases the ring's diameter in jumps of at least 1 millimeter (mm).

It is hence another scope of the present invention to disclose the method, wherein the increase/decrease of the ring's diameter can be set by means of said actuator.

It is hence another scope of the present invention to disclose the method, further comprising a step of providing said feedback mechanism and said inner mechanism encapsulated in a protective insulating biocompatible material.

It is hence another scope of the present invention to disclose the method wherein said computer further comprises instructions executable by said microprocessor to: actuating said opening/closing mechanism by means of said actuator therefore decreasing said diameter D of said split ring to a specified diameter, if said at least one pressure sensor measures a great increase in pressure; locking said opening/closing mechanism until further notice; thereby actuating as a rape salvation device.

It is hence a scope of the present invention to disclose a method for treating a patient suffering from pelvic organ prolapse and/or stress incontinence and provide real-time adjustments to the treatment, said method comprising the steps of: acquiring an adjustable vaginal pessary comprising an inner mechanism, an outer envelope, an actuator and a feedback mechanism: said inner mechanism comprises: a split ring main body having a diameter D; an opening/closing mechanism interconnected to said split ring; said outer envelope comprises: a biocompatible material envelope; a sleeve made of biocompatible material; said feedback mechanism comprises: at least one pressure sensor; at least one computer comprising at least one microprocessor, at least one memory, at least one input/output (I/O), at least one wireless adaptor, at least one transmitter; said at least one computer interconnected to said at least one pressure sensor; at least one motor interconnected to said at least one computer; at least one battery interconnected to said at least one pressure sensor, said at least one computer and said at least one motor; at least one pessary adjusting mechanism interconnected to said at least one motor; inserting said adjustable vaginal pessary into the vaginal tract; adjusting said adjustable vaginal pessary by actuating said opening/closing mechanism by means of said actuator therefore increasing/decreasing said diameter D of said split ring until it meets the necessities of said patient; wherein during said step of adjusting said adjustable vaginal pessary by actuating said opening/closing mechanism said split ring preserves a circular geometry; setting said feedback mechanism by means of said actuator.

It is hence another scope of the present invention to disclose the method, wherein between said step (b) and said step (c) further comprises a step of inserting said actuator in said sleeve in order to reach said opening/closing mechanism.

It is hence another scope of the present invention to disclose the method, wherein said step of adjusting said adjustable vaginal pessary by actuating said opening/closing mechanism does not increase or decrease the overall volume of said pessary.

It is hence another scope of the present invention to disclose the method, wherein said inner mechanism comprises pneumatic means for adjusting said ring.

It is hence another scope of the present invention to disclose the method, wherein said inner mechanism comprises hydraulic means for adjusting said ring.

It is hence another scope of the present invention to disclose the method, wherein said inner mechanism comprises mechanical means for adjusting said ring.

It is hence another scope of the present invention to disclose the method, wherein said mechanical means is a ratchet mechanism.

It is hence another scope of the present invention to disclose the method, wherein said mechanical means is a telescoping mechanism.

It is hence another scope of the present invention to disclose the method, wherein said at least one pressure sensor is selected from the group consisting of: absolute pressure sensor, gauge pressure sensor, vacuum pressure sensor, differential pressure sensor, sealed pressure sensor, and any combination thereof.

It is hence another scope of the present invention to disclose the method, wherein said at least one pressure sensor is selected from the group of pressure-sensing technology consisting of: piezoresistive strain gauge, capacitive, electromagnetic, piezoelectric, optical, potentiometric, resonant, thermal, ionization, and any combination thereof.

It is hence another scope of the present invention to disclose the method, wherein said at least one memory further comprises a program recorded therein to be executed by said at least one microprocessor.

It is hence another scope of the present invention to disclose the method, wherein said at least one pessary adjusting mechanism is selected from the group consisting of pneumatic, hydraulic, mechanical, ratchet, telescoping.

It is hence another scope of the present invention to disclose the method, wherein said battery is a rechargeable battery.

It is hence another scope of the present invention to disclose the method, wherein said rechargeable battery is a kinetic rechargeable battery.

It is hence another scope of the present invention to disclose the method, wherein the actions made by said feedback mechanism are programmable.

It is hence another scope of the present invention to disclose the method, wherein the programming is done physically via a digital handle or wirelessly via a smartphone, tablet or a dedicated remote control.

It is hence another scope of the present invention to disclose the method, wherein said actuator is a digital actuator.

It is hence another scope of the present invention to disclose the method, wherein said step of setting said feedback mechanism is done physically via a digital handle or wirelessly via a smartphone, tablet or a dedicated remote control.

It is hence another scope of the present invention to disclose the method, wherein said feedback mechanism changes the configuration of said adjustable pessary in about one second.

It is hence another scope of the present invention to disclose the method, wherein said feedback mechanism increases/decreases the ring's diameter in jumps of at least 1 millimeter (mm).

It is hence another scope of the present invention to disclose the method, wherein the increase/decrease of the ring's diameter can be set by means of said actuator.

It is hence another scope of the present invention to disclose the method, further comprising a step of providing said feedback mechanism and said inner mechanism encapsulated in a protective insulating biocompatible material.

It is hence another scope of the present invention to disclose the method wherein said computer further comprises instructions executable by said microprocessor to: actuating said opening/closing mechanism by means of said actuator therefore decreasing said diameter D of said split ring to a specified diameter, if said at least one pressure sensor measures a great increase in pressure; locking said opening/closing mechanism until further notice; thereby actuating as a rape salvation device.

It is hence a scope of the present invention to disclose a method for treating a patient suffering from pelvic organ prolapse and/or stress incontinence and provide real-time adjustments to the treatment, said method comprising the steps of: acquiring an adjustable vaginal pessary comprising an inner mechanism, an outer envelope, an actuator and a feedback mechanism: said inner mechanism comprises: a split ring main body having a diameter D; an opening/closing mechanism interconnected to said split ring; a reversible pivot mechanism interconnected to said split ring; said outer envelope comprises: a biocompatible material envelope; a sleeve made of biocompatible material; said feedback mechanism comprises: at least one pressure sensor; at least one computer comprising at least one microprocessor, at least one memory, at least one input/output (I/O), at least one wireless adaptor, at least one transmitter; said at least one computer interconnected to said at least one pressure sensor; at least one motor interconnected to said at least one computer; at least one battery interconnected to said at least one pressure sensor, said at least one computer and said at least one motor; at least one pessary adjusting mechanism interconnected to said at least one motor; inserting said adjustable vaginal pessary into the vaginal tract; adjusting said adjustable vaginal pessary by actuating said opening/closing mechanism by means of said actuator therefore increasing/decreasing said diameter D of said split ring until it meets the necessities of said patient; wherein during said step of adjusting said adjustable vaginal pessary by actuating said opening/closing mechanism said split ring preserves a circular geometry; further wherein during said step of adjusting said adjustable vaginal pessary by actuating said reversible pivot mechanism, said reversible pivot mechanism confers and preserves an angle to said split ring; setting said feedback mechanism by means of said actuator.

It is hence another scope of the present invention to disclose the method, wherein said actuator actuates aid opening/closing mechanism and said reversible pivot mechanism independently.

It is hence another scope of the present invention to disclose the method, wherein between said step (b) and said step (c) further comprises a step of inserting said actuator in said sleeve in order to reach said opening/closing mechanism and said reversible pivot mechanism.

It is hence another scope of the present invention to disclose the method, wherein said step of adjusting said adjustable vaginal pessary by actuating said opening/closing mechanism does not increase or decrease the overall volume of said pessary.

It is hence another scope of the present invention to disclose the method, wherein said inner mechanism comprises pneumatic means for adjusting said ring.

It is hence another scope of the present invention to disclose the method, wherein said inner mechanism comprises hydraulic means for adjusting said ring.

It is hence another scope of the present invention to disclose the method, wherein said inner mechanism comprises mechanical means for adjusting said ring.

It is hence another scope of the present invention to disclose the method, wherein said mechanical means is a ratchet mechanism.

It is hence another scope of the present invention to disclose the method, wherein said mechanical means is a telescoping mechanism.

It is hence another scope of the present invention to disclose the method, wherein at least one pressure sensor is selected from the group consisting of: absolute pressure sensor, gauge pressure sensor, vacuum pressure sensor, differential pressure sensor, sealed pressure sensor, and any combination thereof.

It is hence another scope of the present invention to disclose the method, wherein at least one pressure sensor is selected from the group of pressure-sensing technology consisting of: piezoresistive strain gauge, capacitive, electromagnetic, piezoelectric, optical, potentiometric, resonant, thermal, ionization, and any combination thereof.

It is hence another scope of the present invention to disclose the method, wherein said at least one memory further comprises a program recorded therein to be executed by said at least one microprocessor.

It is hence another scope of the present invention to disclose the method, wherein said at least one pessary adjusting mechanism is selected from the group consisting of pneumatic, hydraulic, mechanical, ratchet, telescoping.

It is hence another scope of the present invention to disclose the method, wherein said battery is a rechargeable battery.

It is hence another scope of the present invention to disclose the method, wherein said rechargeable battery is a kinetic rechargeable battery.

It is hence another scope of the present invention to disclose the method, wherein the actions made by said feedback mechanism are programmable.

It is hence another scope of the present invention to disclose the method, wherein the programming is done physically via a digital handle or wirelessly via a smartphone, tablet or a dedicated remote control.

It is hence another scope of the present invention to disclose the method, wherein said actuator is a digital actuator.

It is hence another scope of the present invention to disclose the method, wherein said step of setting said feedback mechanism is done physically via a digital handle or wirelessly via a smartphone, tablet or a dedicated remote control.

It is hence another scope of the present invention to disclose the method, wherein said feedback mechanism changes the configuration of said adjustable pessary in about one second.

It is hence another scope of the present invention to disclose the method, wherein said feedback mechanism increases/decreases the ring's diameter in jumps of at least 1 millimeter (mm).

It is hence another scope of the present invention to disclose the method, wherein the increase/decrease of the ring's diameter can be set by means of said actuator.

It is hence another scope of the present invention to disclose the method, further comprising a step of providing said feedback mechanism and said inner mechanism encapsulated in a protective insulating biocompatible material.

It is hence another scope of the present invention to disclose the method wherein said computer further comprises instructions executable by said microprocessor to: actuating said opening/closing mechanism by means of said actuator therefore decreasing said diameter D of said split ring to a specified diameter, if said at least one pressure sensor measures a great increase in pressure; locking said opening/closing mechanism until further notice; thereby actuating as a rape salvation device.

It is hence a scope of the present invention to disclose an adjustable vaginal pessary adapted to provide real-time adjustments by means of a feedback mechanism in order to provide support to vaginal walls of a patient comprising: at least one computer comprising at least one microprocessor, at least one memory, at least one input/output (I/O), at least one wireless adaptor, at least one transmitter; said at least one computer interconnected to said at least one pressure sensor; at least one motor interconnected to said at least one computer; at least one battery interconnected to said at least one computer and said at least one motor; at least one pessary adjusting mechanism interconnected to said at least one motor; wherein said pessary preserves a circular geometry during said adjustments.

It is hence another scope of the present invention to disclose the adjustable vaginal pessary, wherein said at least one memory further comprises a program recorded therein to be executed by said at least one microprocessor.

It is hence another scope of the present invention to disclose the adjustable vaginal pessary, wherein said at least one pessary adjusting mechanism is selected from the group consisting of pneumatic, hydraulic, mechanical, ratchet, telescoping.

It is hence another scope of the present invention to disclose the adjustable vaginal pessary, wherein said battery is a rechargeable battery.

It is hence another scope of the present invention to disclose the adjustable vaginal pessary, wherein said rechargeable battery is a kinetic rechargeable battery.

It is hence another scope of the present invention to disclose the adjustable vaginal pessary, wherein said adjustments are programmable.

It is hence another scope of the present invention to disclose the adjustable vaginal pessary, wherein the programming is done physically via a digital handle or wirelessly via a smartphone, tablet or a dedicated remote control.

It is hence another scope of the present invention to disclose the adjustable vaginal pessary, wherein said changing configuration of said adjustable pessary occurs in about one second.

It is hence another scope of the present invention to disclose the adjustable vaginal pessary, wherein said adjustments increases/decreases the ring's diameter in jumps of at least 1 millimeter (mm).

It is hence another scope of the present invention to disclose the adjustable vaginal pessary, wherein the increase/decrease of the ring's diameter can be set by means of said actuator.

It is hence another scope of the present invention to disclose the adjustable vaginal pessary, wherein said feedback mechanism is encapsulated in a protective insulating biocompatible material.

It is hence another scope of the present invention to disclose the adjustable vaginal pessary, wherein said pessary is used as a rape salvation device.

### BRIEF DESCRIPTION OF THE FIGURES

FIG. 1 showing a schematic representation of a method of operation of one embodiment of the present invention;

FIG. 2 is a schematic representation, not in scale, of the inner mechanism of one embodiment of the present invention;

FIGS. 3 a-c are a schematic representation, not in scale, of the opening/closing mechanism of one embodiment of the present invention;

FIG. 4 is a schematic representation, not in scale, of the actuator of one embodiment of the present invention;

FIG. 5 is a schematic representation, not in scale, of the folding mechanism of one embodiment of the present invention;

FIGS. 6 a-b are schematic representations, not in scale, of the folding mechanism of one embodiment of the present invention;

FIG. 7 is a schematic representation, not in scale, of the folding mechanism of one embodiment of the present invention;

FIG. 8 is a schematic representation, not in scale, of the operation of the actuator of one embodiment of the present invention;

FIG. 9 is a schematic representation of one embodiment of the present invention showing the pessary comprising pressure sensors;

FIG. 10 is a schematic flowchart of the connection between the components of the feedback mechanism of one embodiment of the present invention;

FIG. 11 is a schematic flowchart of the method of activation of the feedback mechanism;

FIG. 12 is a schematic representation, not in scale, of several actuators of one embodiment of the present invention;

FIG. 13 is a schematic flowchart of the connection between the components of the feedback mechanism of another embodiment of the present invention;

FIG. 14 is a schematic flowchart of the method of activation of the feedback mechanism;

FIG. 15 is a schematic representation, not in scale, of several actuators of one embodiment of the present invention;

FIG. 16 is a schematic representation of one embodiment of the whole invention comprising pressure sensors;

FIG. 17 is a schematic representation of another embodiment the whole invention without pressure sensors.

FIG. 18 is a chart showing the pressure values (calculated in mmHg) calculated for different type of activities.

FIG. 19 is an exemplary embodiment of the inner mechanism of the pessary of the present invention.

FIG. 20 is another exemplary embodiment of the inner mechanism of the pessary of the present invention.

### DETAIL DESCRIPTION OF THE PREFERRED EMBODIMENTS

The following description is provided, so as to enable any person skilled in the art to make use of said invention and sets forth the best modes contemplated by the inventor of carrying out this invention. Various modifications, however, are adapted to remain apparent to those skilled in the art, since the generic principles of the present invention have been defined specifically to provide a vaginal pessary used for treating pelvic genital organ prolapse and female urinary distress, especially stress incontinence.

The present invention discloses a novel feedback system for a vaginal pessary which comprises at least one sensor coupled to an electronic unit which then operates at least one actuator following a predetermined program in order to provide real-time support to vaginal walls of a patient suffering of pelvic genital organ prolapse and female urinary distress, especially stress incontinence.

It is a scope of the present invention to provide a novel feedback system for a vaginal pessary which confers yet more degrees of freedom.

The term “computer” or “computing unit” refers hereinafter to a device that can be instructed to carry out an arbitrary set of arithmetic or logical operations automatically. Conventionally, a computer consists of at least one processing element, typically a central processing unit (CPU), and some form of memory. The processing element carries out arithmetic and logical operations, and a sequencing and control unit can change the order of operations in response to stored information.

The term “motor” or “activator unit” refers hereinafter to a device designed to convert one form of energy into mechanical energy.

The term “pressure” refers hereinafter to the force applied perpendicular to the surface of an object per unit area over which that force is distributed. Gauge pressure (also spelled gage pressure) is the pressure relative to the ambient pressure. Various units are used to express pressure. Some of these derive from a unit of force divided by a unit of area; the SI unit of pressure, the pascal (Pa), for example, is one newton per square meter; similarly, the pound-force per square inch (psi) is the traditional unit of pressure in the imperial and US customary systems. Pressure may also be expressed in terms of standard atmospheric pressure; the atmosphere (ATM) is equal to this pressure and the torr is defined as 1/760 of this. Manometric units such as the centimeter of water, millimeter of mercury, and inch of mercury are used to express pressures in terms of the height of column of a particular fluid in a manometer.

For the matters related to this application, the units of pressure are calculated following the following conversion units:

| | |
|---|---|
| 1 cm H₂O | =98.0665 pascals |
| (conventional) | |
| | =0.01 meter water (m H₂O), |
| | meter water column (m.wc) |
| | or meter water gauge (m wg) |
| | =10 mm wg |
| | ≈0.980665 mbar or hPa |
| | ≈0.39370 in H₂O |
| | ≈0.000967838 atm |
| | ≈0.73556 torr |
| | ≈0.735559 mm Hg |
| | ≈0.0289590 in Hg |
| | ≈0.0142233 psi |

Referring now to FIG. 1 showing in general the mechanism of action of the pessary. The pessary is an adjustable pessary in its circumference, without changing the volume of the pessary. The circumference of the pessary changes in all directions so to keep the circular configuration and only changing the diameter of the ring.

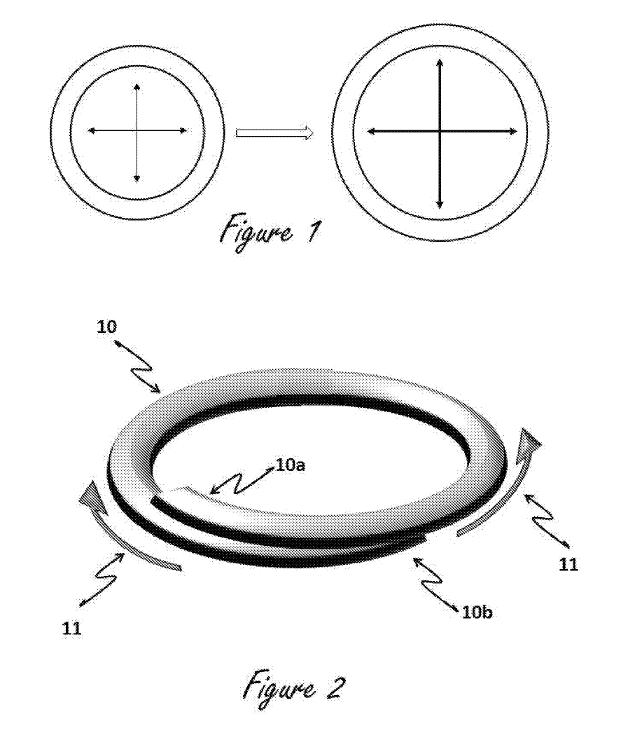

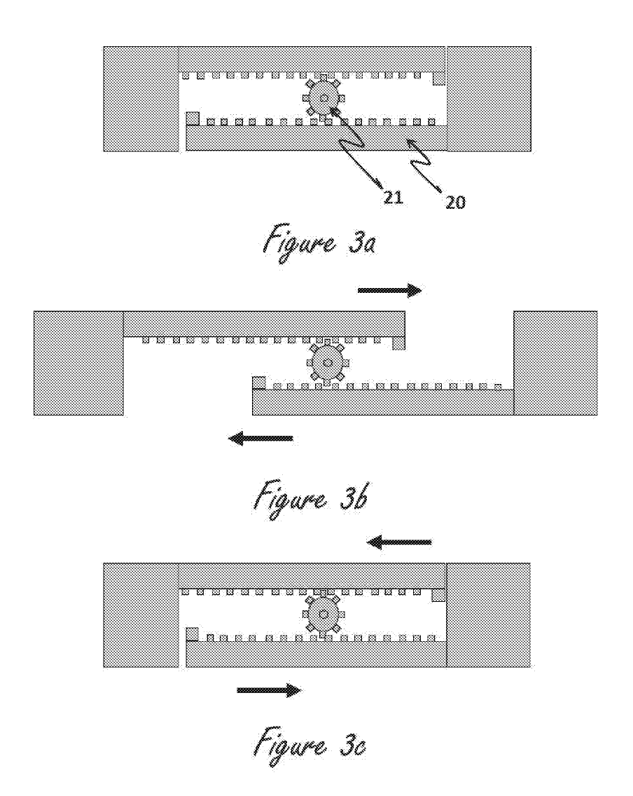

Referring now to FIG. 2 showing the principle behind the mechanism of action of the pessary (not in scale) when said pessary changes diameter without changing its overall volume. The internal mechanism of the pessary is built in a form of a keyring or “split ring” 10 which comprises an overlapping part and said split ring ends in two extremities 10a and 10b. In order to increase the diameter of the pessary, the split ring opens in the direction of the arrows 11. This kind of movement causes the aperture of the ring while preserving its circular form.

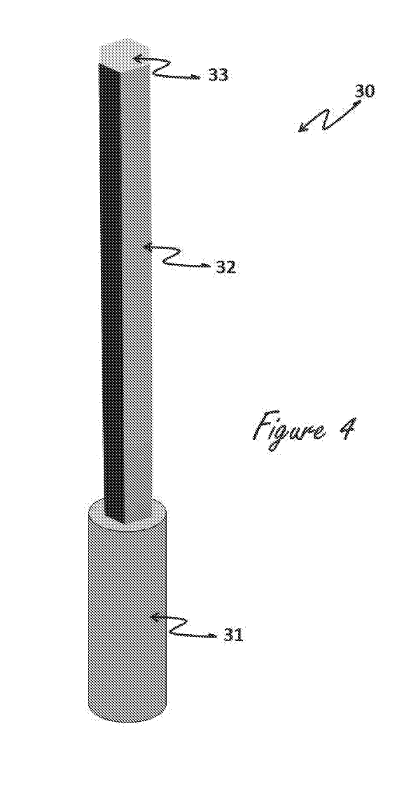

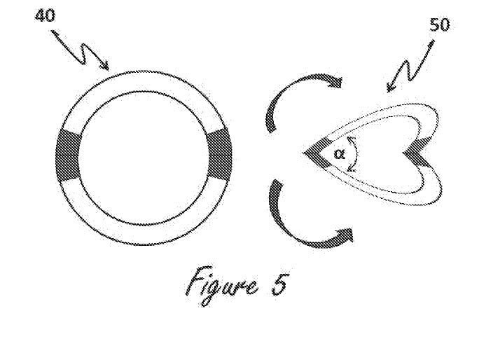

Referring now to FIG. 3 showing an embodiment of the sliding mechanism of the split ring. In this embodiment, the sliding mechanism is configured as a rack 20 and pinion 21 gear. FIG. 3a shows the mechanism in its closed position. The gear comprises at its center an aperture in which the actuator 30 (See FIG. 4) is inserted. FIG. 3 b shows the mechanism in its partially open configuration. Once the gear is operated with the actuator 30 (actuator not shown), the two parts of the split ring move in the direction of the rows. FIG. 3c shows the mechanism in its closed position again, after being closed by the actuator 30.

It is obvious that similar mechanism of action can be used, the aforementioned mechanism was used as an example only and it does not mean to limit the invention.

An important feature of the sliding mechanism is that it enables movement without providing unnecessary volume to the overall volume of the pessary. Another important feature is that provides the user the possibility of changing the diameter without the need to take out the pessary or replacing the pessary with another with different diameter.

Therefore, in all the embodiment of the present invention, the pessary can be used 24 hours a day or for as long as needed.

Referring now to FIG. 4 showing one embodiment of the actuator 30 of the present invention. The actuator 30 comprises a handle 31 which is interconnected to a long pin 32. At the end of said pin there is the part 33 that interconnects with the sliding mechanism. In this embodiment, the pin and the end part have a hexagon form, like a Hex-L key.

It is obvious that different forms or mechanism can be used, the aforementioned hexagon mechanism was used as an example only and it does not mean to limit the invention.

In several other embodiments, the actuator can be configured differently in order to respond to specifics necessities of the user. Ergonomic features are added to the handle, different configurations of the long pin are included in the embodiments of the present application and several other mechanisms of attachment, known in the art, to the internal mechanism of the pessary are also included in the embodiments of the present application.

Referring now to FIG. 5 showing a second degree of freedom of the pessary of the present invention. Beside the possibility of opening the diameter of the pessary while preserving a circular geometry, the pessary of the present invention comprises a reversibly mechanism that allows the pessary to move from a straight configuration 40 to folded configuration 50, therefore acquiring an angle α (alpha), and keep said angle as long as is needed.

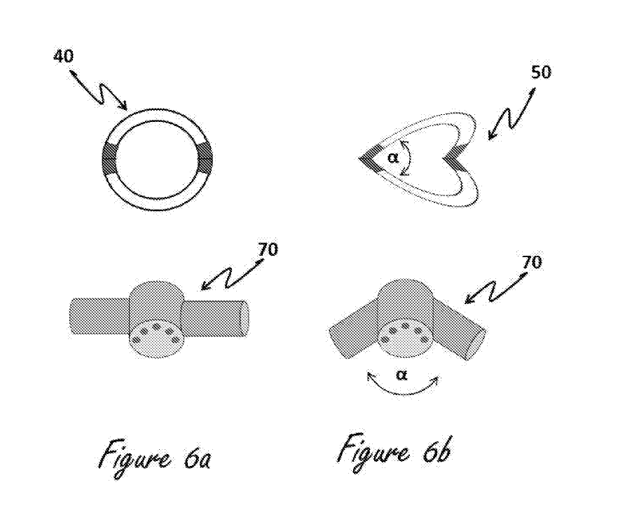

Referring now to FIG. 6 showing an embodiment of the folding mechanism 70 of the present invention. The folding mechanism works like an elbow and includes, in one embodiment of the present invention, a push button latch mechanism. The push button latch mechanism comprises at least one orifice and it can contain any number of orifices as necessary. In this example, the push button latch mechanism comprises five orifices as a way of example. The first orifice keeps the arms of the folding mechanism in a straight position. Each following orifice confers the ring with an angle in an increasing manner. FIG. 6a shows the ring in its straight configuration 40 together with the folding mechanism. FIG. 6b shows an example where the ring is in a folded position 50 having an angle α (alpha) and the folding mechanism that confers said angle.

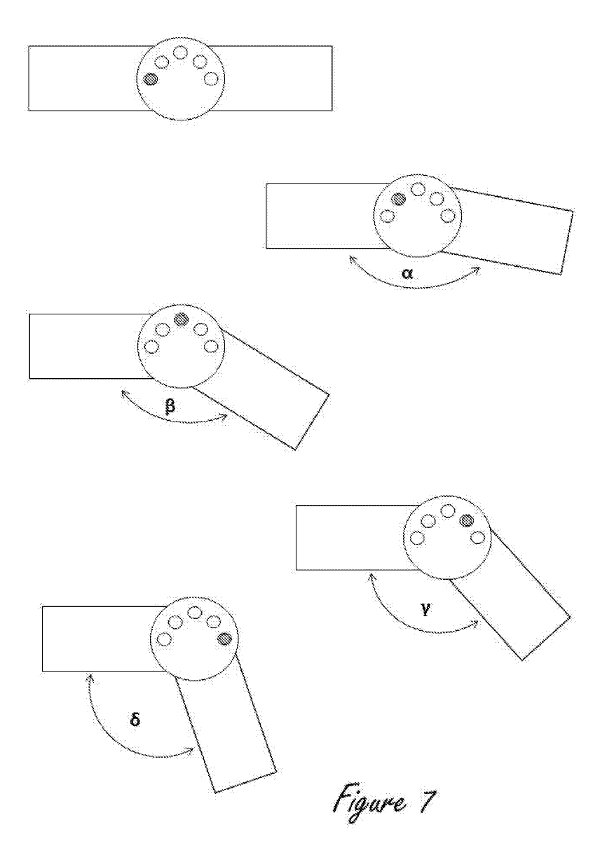

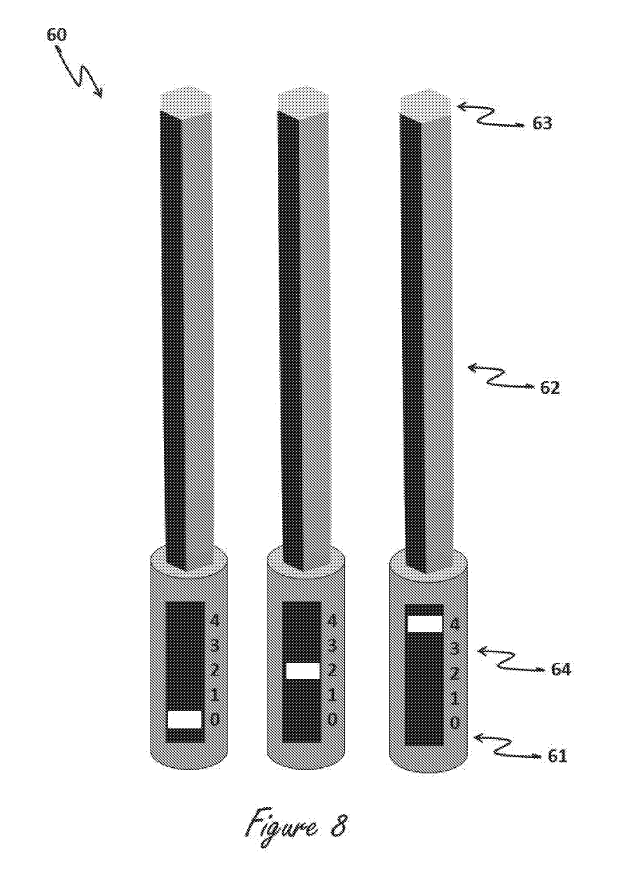

Referring now to FIG. 7 showing, as an exemplary manner, the possible positions that the foldable ring can achieve by using different orifices of the folding mechanism. It can be seen that different orifices of the push button latch mechanism confer different angles to the ring (angles α, β, γ and δ).

Referring now to FIG. 8 showing another embodiment of the actuator 60 of the present invention. The actuator 60 comprises a handle 61 which is interconnected to a long pin 62. At the end of said pin there is the part 63 that interconnects with the sliding mechanism. In this embodiment, the pin and the end part have a hexagon form, like a Hex-L key. Furthermore, actuator 60 comprises the actuation mechanism 64 for the foldable option of the present invention. In this example, the actuation mechanism comprises the numbers zero to four, where zero is the straight configuration and four is the maximum foldable position. The user can move the actuation mechanism from each one of the positions by pressing the button and moving it to the desired position.

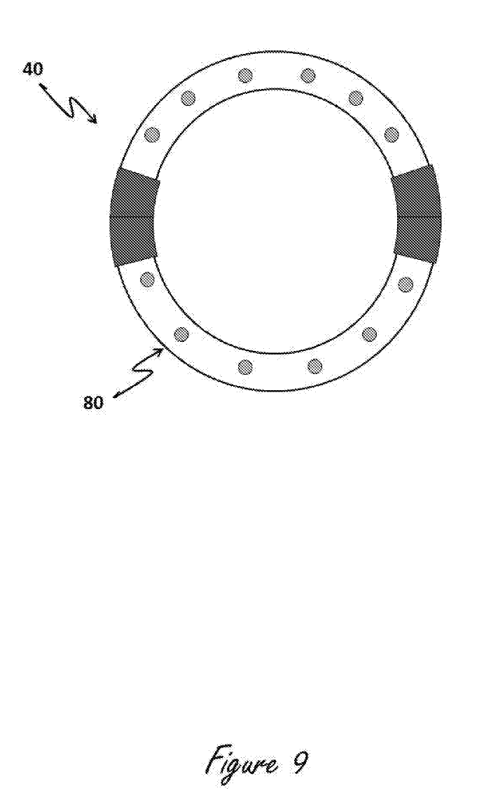

Referring now to FIG. 9 showing an embodiment of the present invention. The pessary 40 comprises several pressure sensors 80 through the circumference of the pessary.

A pressure sensor measures pressure, typically of gases, liquids or forces. Pressure is an expression of the force required to stop a fluid from expanding, and is usually stated in terms of force per unit area. A pressure sensor usually acts as a transducer; it generates a signal as a function of the pressure imposed. For the purposes of this invention, such a signal is electrical (https://en.wikipedia.org/wiki/Pressure_sensor—incorporated herein as reference).

Pressure sensors are used for control and monitoring in thousands of everyday applications. Pressure sensors can also be used to indirectly measure other variables such as fluid/gas flow, speed, water level, and altitude. Pressure sensors can alternatively be called pressure transducers, pressure transmitters, pressure senders, pressure indicators, piezometers and manometers, among other names.

Pressure sensors can vary drastically in technology, design, performance, application suitability and cost.

There is also a category of pressure sensors that are designed to measure in a dynamic mode for capturing very high-speed changes in pressure. These sensors are commonly manufactured out of piezoelectric materials such as quartz.

Some pressure sensors function in a binary (off/on) manner, i.e., when pressure is applied to a pressure sensor, the sensor acts to complete or break an electrical circuit. These types of sensors are also known as a pressure switch.

### Types of Pressure Measurements

Pressure sensors can be classified in terms of pressure ranges they measure, temperature ranges of operation, and most importantly the type of pressure they measure. Pressure sensors are variously named according to their purpose, but the same technology may be used under different names.
Absolute Pressure Sensor

This sensor measures the pressure relative to perfect vacuum.
Gauge Pressure Sensor

This sensor measures the pressure relative to atmospheric pressure. A tire pressure gauge is an example of gauge pressure measurement; when it indicates zero, then the pressure it is measuring is the same as the ambient pressure.
Vacuum Pressure Sensor

This term can cause confusion. It may be used to describe a sensor that measures pressures below atmospheric pressure, showing the difference between that low pressure and atmospheric pressure (i.e. negative gauge pressure), but it may also be used to describe a sensor that measures low pressure relative to perfect vacuum (i.e. absolute pressure).
Differential Pressure Sensor

This sensor measures the difference between two pressures, one connected to each side of the sensor. Differential pressure sensors are used to measure many properties, such as pressure drops across oil filters or air filters, fluid levels (by comparing the pressure above and below the liquid) or flow rates (by measuring the change in pressure across a restriction). Technically speaking, most pressure sensors are really differential pressure sensors; for example, a gauge pressure sensor is merely a differential pressure sensor in which one side is open to the ambient atmosphere.
Sealed Pressure Sensor

This sensor is similar to a gauge pressure sensor except that it measures pressure relative to some fixed pressure rather than the ambient atmospheric pressure (which varies according to the location and the weather).

### Pressure-Sensing Technology

There are two basic categories of pressure sensors: force collectors and others.

Force collector types: These types of electronic pressure sensors generally use a force collector (such a diaphragm, piston, bourdon tube, or bellows) to measure strain (or deflection) due to applied force over an area (pressure).
Piezoresistive Strain Gauge

Uses the piezoresistive effect of bonded or formed strain gauges to detect strain due to applied pressure, resistance increasing as pressure deforms the material. Common technology types are Silicon (Monocrystalline), Polysilicon Thin Film, Bonded Metal Foil, Thick Film, and Sputtered Thin Film. Generally, the strain gauges are connected to form a Wheatstone bridge circuit to maximize the output of the sensor and to reduce sensitivity to errors. This is the most commonly employed sensing technology for general purpose pressure measurement.
Capacitive

Uses a diaphragm and pressure cavity to create a variable capacitor to detect strain due to applied pressure, capacitance decreasing as pressure deforms the diaphragm. Common technologies use metal, ceramic, and silicon diaphragms.
Electromagnetic

Measures the displacement of a diaphragm by means of changes in inductance (reluctance), LVDT, Hall Effect, or by eddy current principle.
Piezoelectric

Uses the piezoelectric effect in certain materials such as quartz to measure the strain upon the sensing mechanism due to pressure. This technology is commonly employed for the measurement of highly dynamic pressures.
Optical

Techniques include the use of the physical change of an optical fiber to detect strain due to applied pressure. A common example of this type utilizes Fiber Bragg Gratings. This technology is employed in challenging applications where the measurement may be highly remote, under high temperature, or may benefit from technologies inherently immune to electromagnetic interference. Another analogous technique utilizes an elastic film constructed in layers that can change reflected wavelengths according to the applied pressure (strain).
Potentiometric

Uses the motion of a wiper along a resistive mechanism to detect the strain caused by applied pressure.

Other types: These types of electronic pressure sensors use other properties (such as density) to infer pressure of a gas, or liquid.
Resonant

Uses the changes in resonant frequency in a sensing mechanism to measure stress, or changes in gas density, caused by applied pressure. This technology may be used in conjunction with a force collector, such as those in the category above. Alternatively, resonant technology may be employed by exposing the resonating element itself to the media, whereby the resonant frequency is dependent upon the density of the media. Sensors have been made of vibrating wire, vibrating cylinders, quartz, and silicon MEMS. Generally, this technology is considered to provide very stable readings over time.
Thermal

Uses the changes in thermal conductivity of a gas due to density changes to measure pressure. A common example of this type is the Pirani gauge.
Ionization

Measures the flow of charged gas particles (ions) which varies due to density changes to measure pressure. Common examples are the Hot and Cold Cathode gauges.

In one embodiment of the present invention the pressure sensor is selected from the group consisting of: absolute pressure sensor, gauge pressure sensor, vacuum pressure sensor, differential pressure sensor, sealed pressure sensor, and any combination thereof

In another embodiment of the present invention the pressure sensor is selected from the group of pressure-sensing technology consisting of piezoresistive strain gauge, capacitive, electromagnetic, piezoelectric, optical, potentiometric, resonant, thermal, ionization, and any combination thereof.

In another embodiment of the present invention the pressure sensor is a digital sensor.

The sensors of the present invention can be distributed equally through the pessary or sporadically distributed. The sensors can be located on one side or on both sides of the pessary.

The sensor of the present invention is connected to a computing unit (referred hereinafter as ‘computer’) having at least one microprocessor, memory, input/output (I/O), wireless adaptors and other features required of a functional computer.

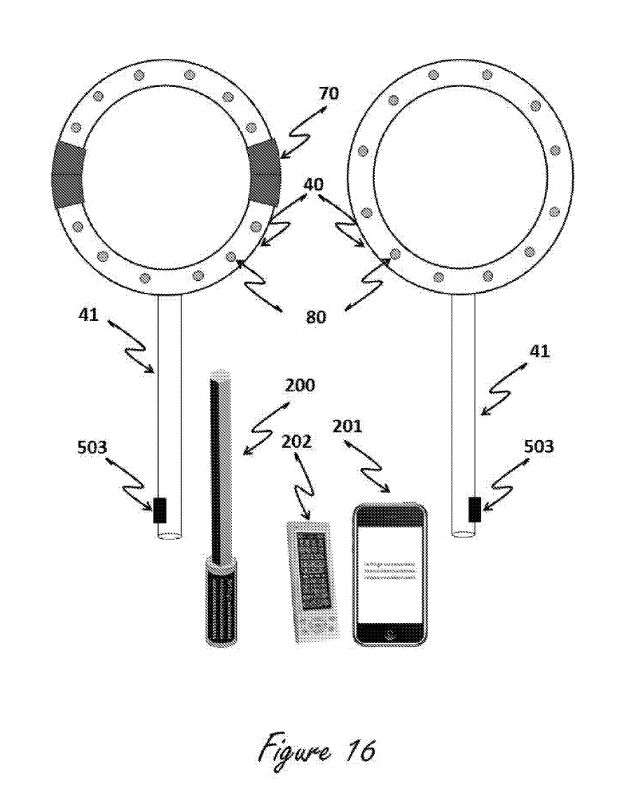

In a preferred embodiment of the present invention, the wireless adaptor can be located at the far end of the sleeve, related to the ring (see 503 in FIG. 16). This allows the wireless adaptor to be outside the body and ensuring the connection with external devices.

The computer is adapted to receive the inputs from the at least one sensor and convert them into operational information.

The computer is connected also to an activator unit (referred hereinafter as ‘motor’) that activates the actuator (i.e. 21 of FIG. 3a), causing the pessary to either open or close, depending on the pressure input received.

In several embodiments of the present invention the ‘computer’ can be a microcomputer, a nanocomputer or any other computing unit which is small enough to be allocated inside the body of the pessary and fully perform.

In several embodiments of the present invention the ‘motor’ can be a micromotor, a nanomotor or any other activator unit which is small enough to be allocated inside the body of the pessary and fully perform.

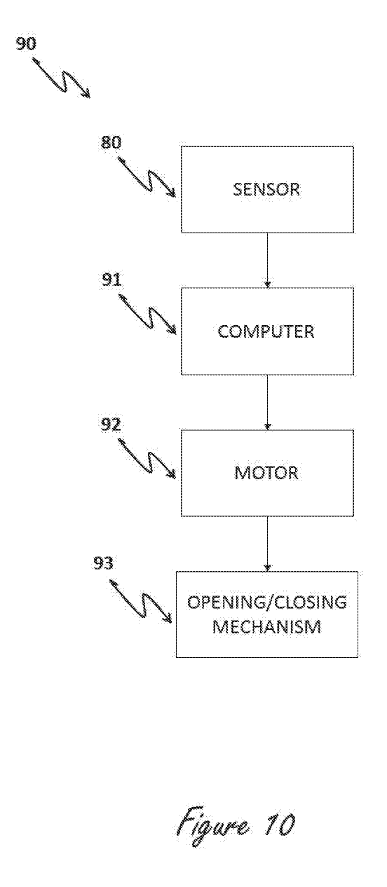

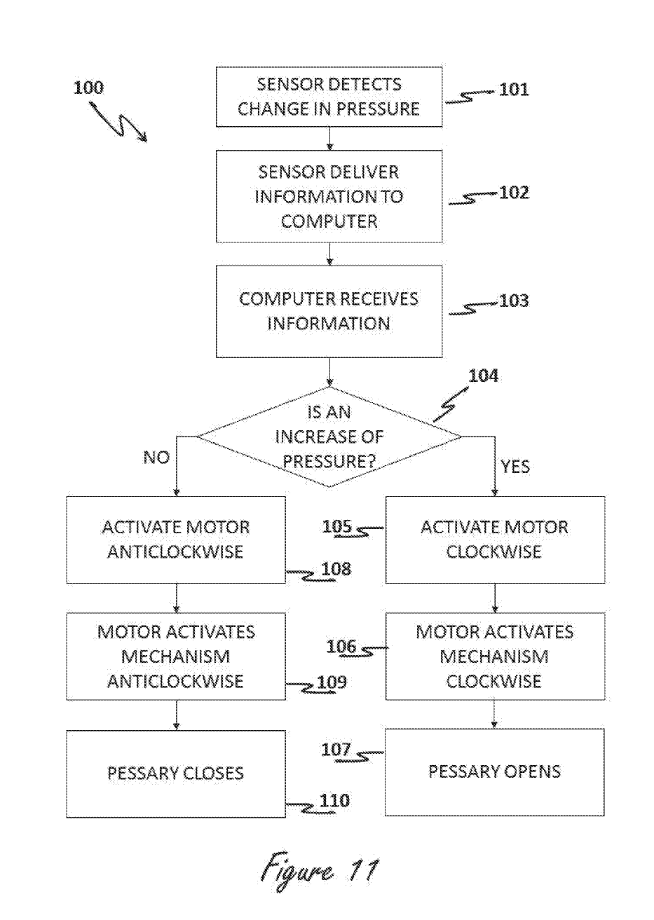

Referring now to FIG. 10 showing a schematic flowchart of the connections between the different parts of the feedback system 90 of the present invention. The sensor 80 is connected to the computer 91, which is connected to a motor 92, which is connected and activates the opening/closing mechanism 93.

Referring now to FIG. 11 showing a schematic flowchart of the method 100 of action of the feedback system of the pessary. Once the pessary is in place and manually adapted by the user, the sensors begin to work. The sensors feel all the time certain amount of pressure due to the pathology. If the difference in the pressure is too high or too low the sensor detects this change 101. The sensor then delivers the information to the computer 102. Once the computer receives the information 103, it analyzes said information to determine whether the change in the pressure is an increment in the pressure or not 104. If the answer is “yes” then the computer activates the motor clockwise 105. This causes the opening/closing mechanism to move clockwise 106, therefore opening the pessary 107 to enable it to stand the pressure while still working and without damaging or causing discomfort to the user. On the other hand, if the answer is “no” then the computer activates the motor anticlockwise 108. This causes the opening/closing mechanism to move anticlockwise 109, therefore closing the pessary 110 to enable it to fit better and work as it supposed to.

It will be obvious to any person skilled in the art that this example is not limiting and the decision algorithm can vary, together with the activation of different actuators depending on their method of activation. Also, the response to variations of pressure can be different between users. An increase of pressure can cause the pessary to either open or close, depending on the condition, and vice versa.

Furthermore, in an embodiment of the present invention, the pressure threshold on which the feedback mechanism is activated can be personalized by user. Also, the level of activation, therefore the level of opening or closing of the pessary, can be personalized as well to fit better the needs of the user.

In an embodiment of the present invention the adjustments made by the feedback mechanism are done in a very short time allowing real-time performance of the device. In an embodiment, the feedback mechanism changes the diameter of the ring in a total time of 2 seconds. In a preferred embodiment, the feedback mechanism changes the diameter of the ring in a total time of 1 second.

In an embodiment of the present invention the feedback mechanism can increase the ring's diameter in jumps of at least 1 millimeter (mm) or any number of millimeters set by default or specifically by the user.

In an embodiment of the present invention, the feedback mechanism can differentiate between a quick change in pressure, like in the case of sneezing, and a long change in pressure, like when performing sport activities.

In an embodiment of the present invention, the settings of the feedback mechanism can be accessed directly via the handle, which in this case will be a digital handle, or wireless using a dedicated application for a smartphone or tablet or a dedicated remote control.

In another embodiment of the present invention, the pessary can be adjusted as well, directly by the user using the same application, without activating the feedback mechanism.

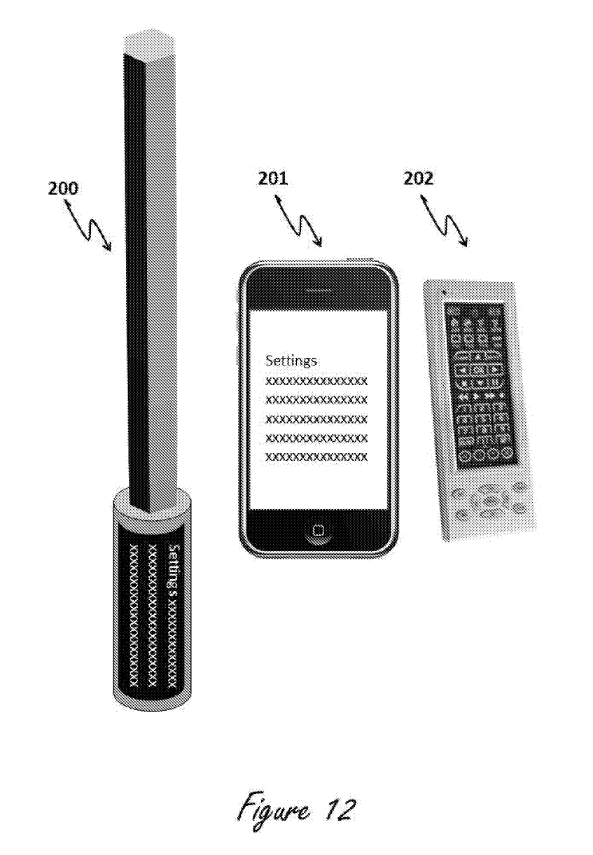

Referring now to FIG. 12 showing a schematic, not in scale, embodiment of the present invention. A digital handle actuator 200 with a touchscreen that enables the user to set personalized parameters related to the pessary. These parameters can be set via a dedicated application on a smartphone 201 or a tablet. Also, the parameters can be set via a dedicated remote control 202.

The electronic method of activating the pessary is also used for the folding mechanism of the pessary. In certain embodiments, the user can decide the level of folding using the electronic handle or the application in the smartphone/tablet. The feedback mechanism can also activate the folding mechanism in case the sensors sense the pressure arriving mainly from the collapsing internal organs of the user.

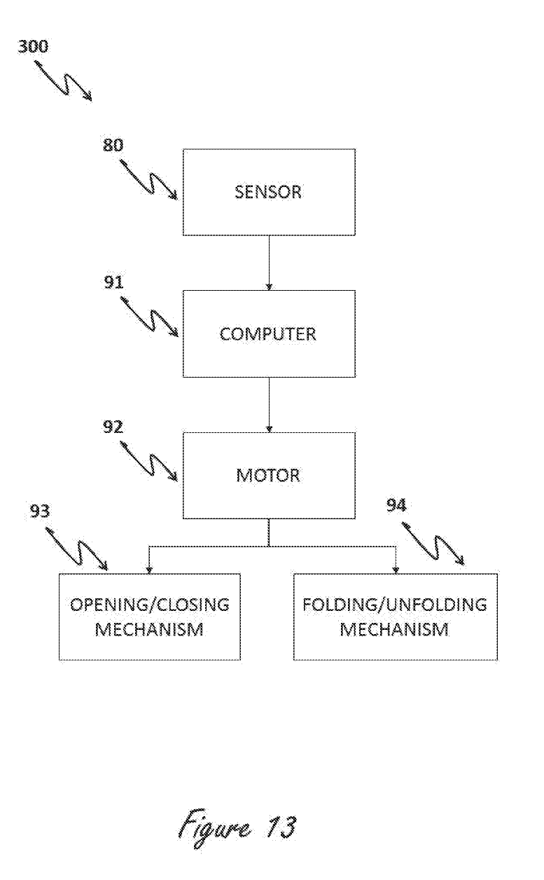

Referring now to FIG. 13 showing another schematic flowchart of the possible connections between the different parts of the feedback system 90 of the present invention. The sensor 80 is connected to the computer 91, which is connected to a motor 92, which is connected and activates the opening/closing mechanism 93 and/or folding/unfolding mechanism 94.

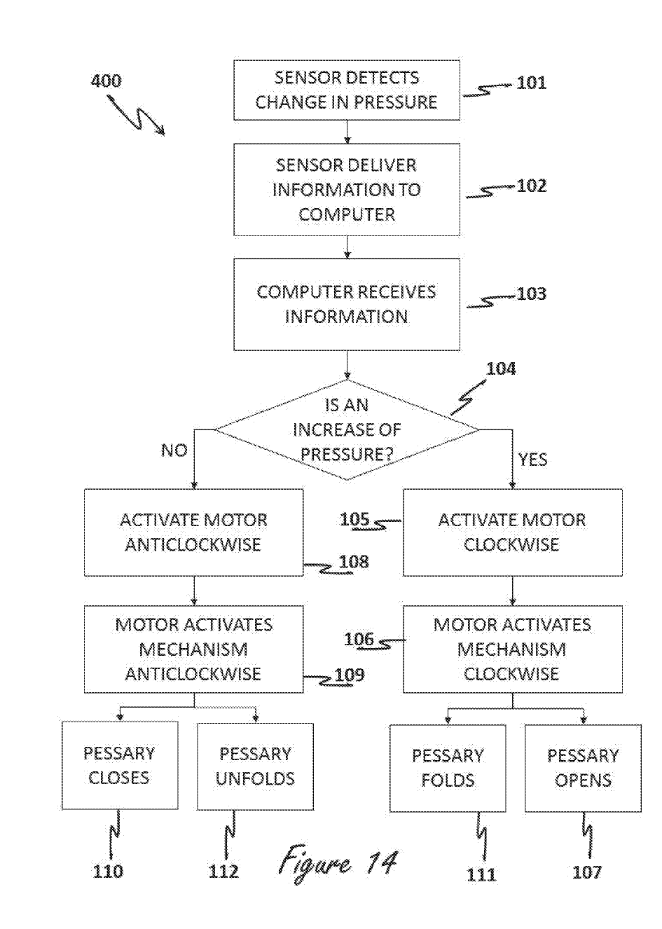

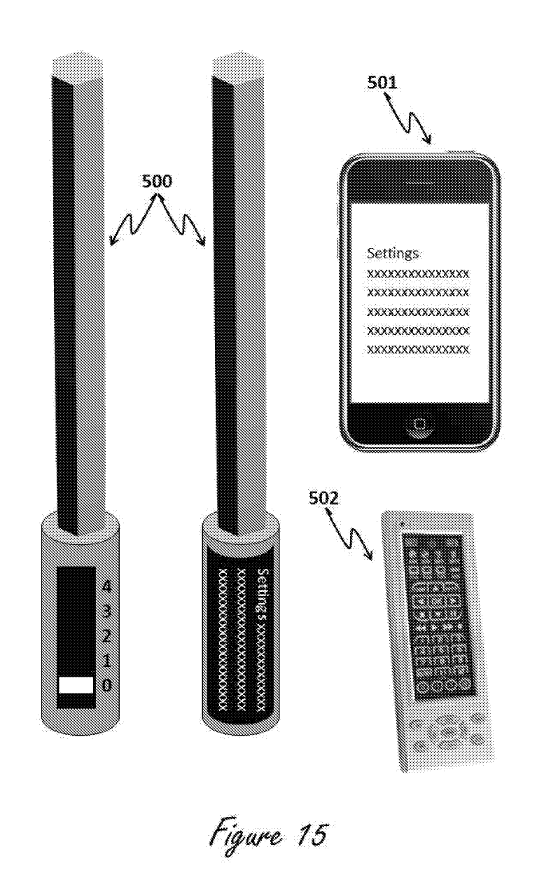

Referring now to FIG. 14 showing a schematic flowchart of the method 100 of action of the feedback system of the pessary. Once the pessary is in place and manually adapted by the user, the sensors begin to work. The sensors feel all the time certain amount of pressure due to the pathology. If the difference in the pressure is too high or too low the sensor detects this change 101. The sensor then delivers the information to the computer 102. Once the computer receives the information 103, it analyzes said information to determine whether the change in the pressure is an increment in the pressure or not 104. If the answer is “yes” then the computer activates the motor clockwise 105. This causes the opening/closing mechanism to move clockwise 106, therefore opening the pessary 107 to enable it to stand the pressure while still working and without damaging or causing discomfort to the user or/and folding the pessary 111 to “lift” further the internal organs. On the other hand, if the answer is “no” then the computer activates the motor anticlockwise 108. This causes the opening/closing mechanism to move anticlockwise 109, therefore closing the pessary 110 to enable it to fit better and work as it supposed to; and/or unfold the pessary 112.

It will be obvious to any person skilled in the art that this example is not limiting and the decision algorithm can vary, together with the activation of different actuators depending on their method of activation. Also, the response to variations of pressure can be different between users. An increase of pressure can cause the pessary to either open or close, depending on the condition, and vice versa.

Furthermore, in an embodiment of the present invention, the pressure threshold on which the feedback mechanism is activated can be personalized by user. Also, the level of activation, therefore the level of opening or closing of the pessary, can be personalized as well to fit better the needs of the user.

In an embodiment of the present invention, the settings of the feedback mechanism can be accessed or directly via the handle, which in this case will be a digital handle, or wireless using a dedicated application for a smartphone or tablet.

In another embodiment of the present invention, the pessary can be adjusted as well, directly by the user using the same application, without activating the feedback mechanism.

Referring now to FIG. 15 showing a schematic, not in scale, embodiment of the present invention. A digital handle actuator 500 with a touchscreen that enables the user to set personalized parameters related to the pessary and on the other side the actuation mechanism comprises the manual folding control. These parameters can also be set via a dedicated application on a smartphone 501 or a tablet. Also, the parameters can be set via a dedicated remote control 502.

The electronic method of activating the pessary is also used for the folding mechanism of the pessary. In certain embodiments, the user can decide the level of folding using the electronic handle or the application in the smartphone/tablet. The feedback mechanism can also activate the folding mechanism in case the sensors sense the pressure arriving mainly from the collapsing internal organs of the user.

Referring now to FIG. 16 showing the whole invention of the present application, the pessary ring 40 with the pressure sensors 80, with or without the folding mechanism 70, the sleeve 41 and the digital actuator 200 or the smartphone 30.

In several embodiments of the present invention another sensor for measuring the at least one vaginal parameter can be added to the pessary. The sensor may be selected from a group consisting of: temperature sensor, pH sensor, humidity sensor, accelerometer, geographical locator (e.g. GPS), and any combination thereof.

The other sensor can also connect to the computer which can transmit at least one parameter to an external device; and a receiver for receiving at least one signal from an external device.

The pessary also comprises a rechargeable battery. The rechargeable battery can be a kinetic rechargeable battery.

In a further embodiment of the present invention, the internal mechanism of the pessary, compressing all the different folding mechanisms, feedback mechanisms, batteries, etc., is encapsulated in a biocompatible insulating material that protects the user from possible chemical spillages or malfunctions of any part of said internal mechanism.

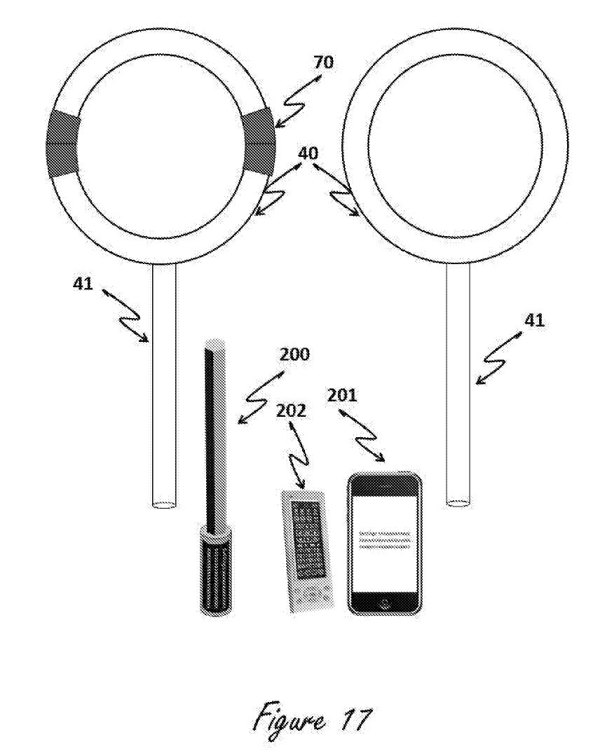

Referring now to FIG. 17 showing another embodiment of the whole invention of the present application, the pessary ring 40 without the pressure sensors with or without the folding mechanism 70, the sleeve 41 and the digital actuator 200 or the smartphone 30 or the dedicated remote control 201. In this case the user can change the diameter of the ring manually at will, without the need of the automated feedback mechanism.

### EXAMPLE 1

In a study made by Kruger J. et al, (intra-abdominal pressure increase in women during exercise: a preliminary study. http://www.ics.org/Abstracts/Publish/134/000537.pdf, incorporated herein as reference), measurements of intra-abdominal pressure were taken using a wireless intra-vaginal pressure device while the subjects performed different tasks.

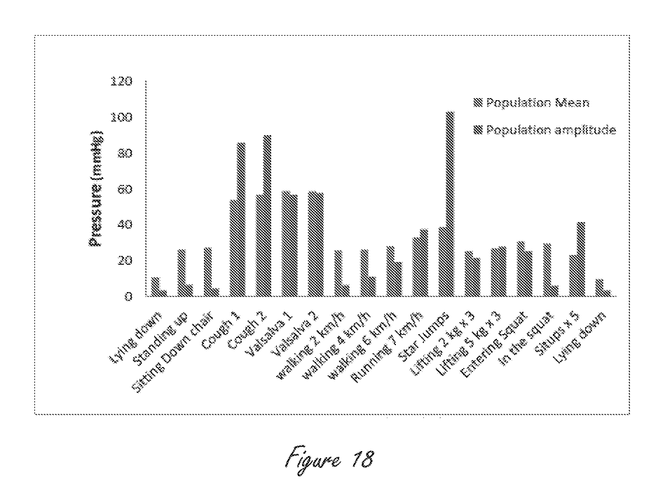

As can be seen in FIG. 18, pressure values (calculated in mmHg) can be calculated for different type of activities.

For the example of the device of the present invention, the values shown in the graph of FIG. 18 will be used for reference.

The feedback device can be set with a normal pressure baseline of about 25 mmHg. Then the thresholds of activation can be set for example at 40 to 45 mmHg. At that point, the ring's diameter is increased by 4 mm. for example.

The sensors of the present invention can perceive little changes in pressure, so in a case of severe organ collapse, the parameters of activation can be more severe. For example, a baseline of about 30 mmHg (due to the collapse) and a threshold of activation at 35 mmHg, opening the ring's diameter 4 mm after that point, for each increment in unit of pressure the ring's diameter will be increased by 2 mm.

It is clear that, once the pressure decreases the feedback mechanism decreases the ring's diameter accordingly.

### EXAMPLE 2

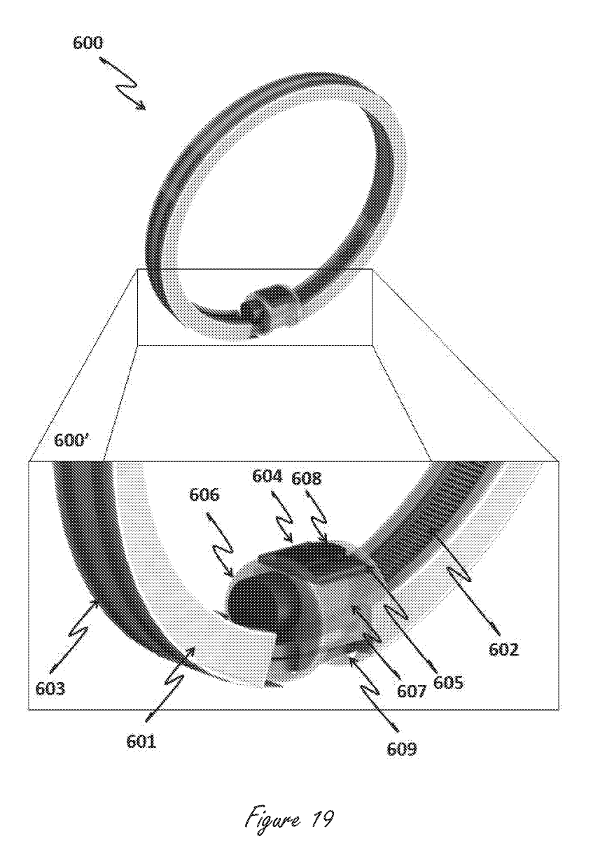

FIG. 19 shows a practical example of the internal mechanism 600 of the pessary of the present invention. As previously described, the system will operate as a metal hose clamp. A zoom-in 600′ of the mechanism is shown. The inner part of the ring will include flexible track 601 with a flexible slotted band 602 within it. While pressure is applied on the ring, a Force Sensitive Resistor (FSR) sensor 603 attached to the circumference of the track 601 will transmit pressure data to the microcontroller 604, located on a printed circuit board (PCB) 605, then activate a motor 606 attached to a worm gear 607, by means of the included battery 608. The whole inner mechanism is closed in a dedicated housing 609 for the safety of the user and the integrity of the mechanism. The band will travel back and forth according to pressure causing the ring to expand or contract accordingly.

### EXAMPLE 3

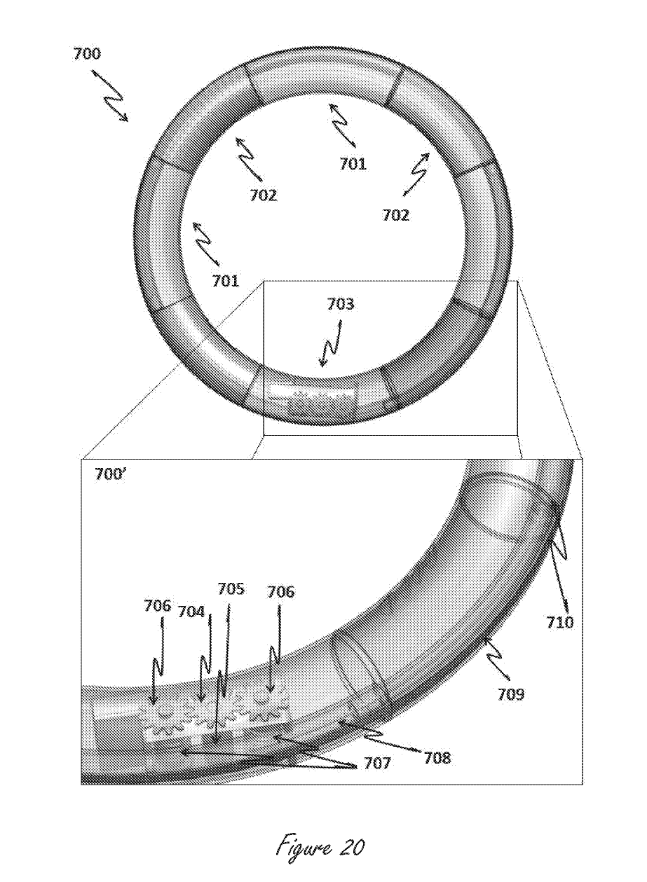

FIG. 20 shows another practical example of the internal mechanism 700 of the pessary of the present invention. The ring includes 4 ring quarters 701 attached with 4 angular bellows 702 between them and expending mechanism 703 based on gears 704 connected to a motor shaft 705 and two other gears 706 connected to shafts, all equally sized, 2 winch drums 707 with a metal wire 708 winding on each drum and wire telescopic ring leads.

When a FSR sensor 709 at the circumference senses pressure, the motor 705 rotates a gear 704 attached to it, causing each of the gears 706 attached to it, on both sides, to spin at the same speed in opposite directions, causing a shaft with the wheels and drums to rotate. A wire 708 wind on the drum is released through angular hoses. Covering each ring quarter, there are larger ones that can be inserted into each other. At each tip of the small hose, the wire is attached to a bead 710. When the wire is released, the bead pushes the small hose causing it to be inserted into the larger hose. Consequently, bellows attached to the leads expand and contract causing the bellows to stretch in an angular motion which causes the aperture opening to expand or contract.

Rape Salvation Device

The diameter and configuration of the adjustable vaginal pessary is adapted to provide real-time protection from a forced intercourse by trapping a non-wanted penis intruder so to deter rapists and/or save a female from an already started rape process; said adjustment/trapping mechanism comprising at least one pressure sensor; at least one computer comprising at least one microprocessor, at least one memory, at least one input/output (I/O), at least one wireless adaptor, at least one transmitter; said at least one computer interconnected to said at least one pressure sensor, at least one motor interconnected to said at least one computer; at least one battery interconnected to said at least one computer and said at least one motor; at least one pessary adjusting mechanism interconnected to said at least one motor; wherein a signal from the said pressure sensor activates automatically the reduction of the internal diameter of the pessary that tightens around the male penis to a pre-defined pressure that is being sensed by the pressure sensor.

When a woman must or intend to be in an area where she may be exposed to a risk of a rape, she may use the intra-vaginal pessary as a rape salvation device. If the woman becomes a victim of a rape attempt the pessary pressure sensor or other adequate sensor will send a signal that will trigger an immediate closing of the pessary to a pre-defined smaller internal diameter that cause the tightening of the pessary around the male penis to such a degree that causes discomfort, mild to great pain, enough to provoke a moment of shock to the man so to allow the woman enough time to escape from the scene. The pessary will open up automatically after a pre-defined lapse of time so to avoid any permanent damage to the penis and/or a necrosis, if enabled by the user. Therefore, the rapist will have to get medical assistance so to be revealed as the rapist.

Based upon the foregoing disclosure, it should now be apparent that the vaginal pessary as described herein will carry out the objects set forth hereinabove. It is, therefore, to be understood that any variations evident fall within the scope of the claimed invention and thus, the selection of specific component elements can be determined without departing from the spirit of the invention herein disclosed and described.

1.-147. (canceled)

148. An adjustable vaginal pessary adapted to provide support to vaginal walls of a patient comprising within:
a. a pessary expanding and contracting mechanism;
b. at least one intravaginal pressure sensor interconnected to said pessary;
c. at least one computer for processing data from said pressure sensor interconnected to said sensor;
wherein said pessary expanding and contracting mechanism is provided with at least one motor for expanding and contracting said reversibly expandable pessary; further wherein said computer is configured to translate said pressure data into programmed instructions for expanding or contracting the configuration of said adjustable pessary in predetermined feedbacked manner.

149. The adjustable vaginal pessary according to claim 148, wherein said pessary further comprises a spring expanding mechanism.

150. The adjustable vaginal pessary according to claim 148, wherein at least one of the following is true:
a. said computer comprises at least one microprocessor, at least one memory, at least one input/output (I/O), at least one wireless adaptor and at least one transmitter;
b. said at least one pressure sensor is selected from the group consisting of: absolute pressure sensor, gauge pressure sensor, vacuum pressure sensor, differential pressure sensor, sealed pressure sensor, and any combination thereof;
c. said at least one pressure sensor is selected from the group of pressure-sensing technology consisting of: piezoresistive strain gauge, capacitive, electromagnetic, piezoelectric, optical, potentiometric, resonant, thermal, ionization, and any combination thereof;
d. said at least one memory further comprises a program recorded therein to be executed by said at least one microprocessor;
e. said at least one pessary adjusting mechanism is selected from the group consisting of pneumatic, hydraulic, mechanical, ratchet, telescoping;
f. expansion of said pessary occurs within an expansion period in a range of 0.1 second to 1 second;
g. contraction of said pessary occurs within a contraction period in a range of 0.5 second to 5 second;
h. said changing configuration of said adjustable pessary occurs in about one second;
i. said feedback mechanism increases/decreases the ring's diameter in jumps of at least 1 millimeter (mm);
j. the increase/decrease of the ring's diameter can be set by means of said actuator;
k. said feedback mechanism is encapsulated in a protective insulating biocompatible material;
l. said pessary is used as a rape salvation device; and
m. said pessary further comprises at least one more sensor selected from a group consisting of: temperature sensor, pH sensor, humidity sensor, accelerometer, geographical locator, and any combination thereof.

151. The adjustable vaginal pessary according to claim 148, wherein said adjustable vaginal pessary further comprises a battery.

152. The adjustable vaginal pessary according to claim 151, wherein one of the following is true:
a. said battery is a rechargeable battery; and
b. said rechargeable battery is a kinetic rechargeable battery.

153. The adjustable vaginal pessary according to claim 148, wherein the actions made by said feedback mechanism are programmable; the programming being done physically via a digital handle or wirelessly via a smartphone, tablet or a dedicated remote control.

154. A feedback mechanism for an adjustable vaginal pessary adapted to provide real-time adjustments in order to provide support to vaginal walls of a patient comprising:
a. at least one pressure sensor interconnected to said vaginal pessary for detecting intravaginal pressure;
b. at least one computer comprising at least one microprocessor, at least one memory, at least one input/output (I/O), at least one wireless adaptor, at least one transmitter; said at least one computer interconnected to said at least one pressure sensor and configured for processing data from said pressure sensor;
c. at least one motor interconnected to said at least one computer;
d. at least one battery interconnected to said at least one pressure sensor, said at least one computer and said at least one motor;
e. at least one pessary adjusting mechanism interconnected to said at least one motor.
wherein said computer is configured to translate said pressure data into programmed instructions for expanding or contracting the configuration of said adjustable pessary in predetermined manner.

155. The feedback mechanism according to claim 154, wherein at least one of the following is true:
a. said at least one pressure sensor is selected from the group consisting of: absolute pressure sensor, gauge pressure sensor, vacuum pressure sensor, differential pressure sensor, sealed pressure sensor, and any combination thereof;
b. said at least one pressure sensor is selected from the group of pressure-sensing technology consisting of: piezoresistive strain gauge, capacitive, electromagnetic, piezoelectric, optical, potentiometric, resonant, thermal, ionization, and any combination thereof;
c. said at least one memory further comprises a program recorded therein to be executed by said at least one microprocessor;
d. said at least one pessary adjusting mechanism is selected from the group consisting of pneumatic, hydraulic, mechanical, ratchet, telescoping;
e. expansion of said pessary occurs within an expansion period in a range of 0.1 second to 1 second;
f. contraction of said pessary occurs within a contraction period in a range of 0.5 second to 5 second;
g. said changing configuration of said adjustable pessary occurs in about one second;
h. said feedback mechanism increases/decreases the ring's diameter in jumps of at least 1 millimeter (mm);
i. the increase/decrease of the ring's diameter can be set by means of said actuator;
j. said feedback mechanism is encapsulated in a protective insulating biocompatible material;
k. said feedback mechanism is used as a rape salvation device; and
l. said pessary further comprises at least one more sensor selected from a group consisting of: temperature sensor, pH sensor, humidity sensor, accelerometer, geographical locator, and any combination thereof.

156. The feedback mechanism according to claim 154, wherein said battery is a rechargeable battery.

157. The feedback mechanism according to claim 156, wherein said rechargeable battery is a kinetic rechargeable battery.

158. The feedback mechanism according to claim 154, wherein the actions made by said feedback mechanism are programmable.

159. The feedback mechanism according to claim 158, wherein the programming is done physically via a digital handle or wirelessly via a smartphone, tablet or a dedicated remote control.

160. An adjustable vaginal pessary adapted to provide real-time adjustments by means of a feedback mechanism in order to provide support to vaginal walls of a patient comprising:
a. at least one computer comprising at least one microprocessor, at least one memory, at least one input/output (I/O), at least one wireless adaptor, at least one transmitter; said at least one computer interconnected to said at least one pressure sensor;
b. at least one motor interconnected to said at least one computer;
c. at least one battery interconnected to said at least one computer and said at least one motor;
d. at least one pessary adjusting mechanism interconnected to said at least one motor. wherein said pessary preserves a circular geometry during said adjustments.

161. The adjustable vaginal pessary according to claim 160, wherein said pessary further comprises a spring expanding mechanism.

162. The adjustable vaginal pessary according to claim 160, wherein at least one of the following is true:
a. said at least one memory further comprises a program recorded therein to be executed by said at least one microprocessor;
b. said at least one pessary adjusting mechanism is selected from the group consisting of pneumatic, hydraulic, mechanical, ratchet, telescoping;
c. expansion of said pessary occurs within an expansion period in a range of 0.1 second to 1 second;
d. contraction of said pessary occurs within a contraction period in a range of 0.5 second to 5 second;
e. said changing configuration of said adjustable pessary occurs in about one second;
f. said feedback mechanism is encapsulated in a protective insulating biocompatible material
g. said pessary is used as a rape salvation device; and
h. said pessary further comprises at least one more sensor selected from a group consisting of: temperature sensor, pH sensor, humidity sensor, accelerometer, geographical locator, and any combination thereof.

163. The adjustable vaginal pessary according to claim 160, wherein said battery is a rechargeable battery.

164. The adjustable vaginal pessary according to claim 162, wherein said rechargeable battery is a kinetic rechargeable battery.

165. The adjustable vaginal pessary according to claim 160, wherein said adjustments are programmable.

166. The adjustable vaginal pessary according to claim 164, wherein the programming is done physically via a digital handle or wirelessly via a smartphone, tablet or a dedicated remote control.

167. The adjustable vaginal pessary according to claim 160, wherein at least one of the following is true:
a. said adjustments increase/decrease the ring's diameter in jumps of at least 1 millimeter (mm); and
b. the increase/decrease of the ring's diameter can be set by means of said actuator.
